(19) United States
(12) Patent Application Publication
(10) Pub. No.: US 2008/0038334 A1
(43) Pub. Date: Feb. 14, 2008

(54) Method for producing and a system for cooling a hot-filled softgel capsule

(76) Inventors: John Zazula, Morris Plains, NJ (US); Reuben O. Zielinski, Belle Mead, NJ (US); Richard Glawson, Beachwood, NJ (US)

Correspondence Address: Fitzpatrick Cella (Catalent), 30 Rockefeller Plaza, New York, NY, 10112, US

(21) Appl. No.: 11/500,719

(22) Filed: Aug. 8, 2006

(51) Int. Cl. A61K 9/64; B29C 39/10

(52) U.S. Cl. 424/456; 264/4

(57) ABSTRACT

A system for producing a hot-filled softgel capsule utilizes a chilled liquid. The chilled liquid is routed through a chilled liquid conveyor tray into a chilled liquid bath. The chilled liquid conveyor tray directs the flowing chilled liquid into a flowing chilled liquid layer. Softgel capsules having a heated fill material are deposited in the flowing chilled liquid layer. The chilled liquid layer cools the capsule by transferring heat from the capsule to the chilled liquid. The flowing chilled liquid layer transports the capsule out of the chilled liquid conveyor tray into a chilled liquid bath. A capsule transfer conveyor transports the capsule out of the chilled liquid bath to a chilled liquid removal device. The chilled liquid removal device removes the chilled liquid from the capsule.

### TECHNICAL FIELD

The present invention generally relates to softgel capsule manufacturing and, more particularly, relates to a method for producing and a system for cooling softgel capsules formed by encapsulating a hot fill material in a film followed by cooling the capsule with a chilled liquid.

### BACKGROUND OF THE INVENTION

Soft capsules generally consist of a shell which is produced, for example, by extending a mixture of gelatin, plasticizer, and water into a thin sheet, film, or band. Capsules formed from such a sheet hold a wide variety of substances. The shell of a soft capsule is typically produced, for example, by adding, to an aqueous gelatin melt, a plasticizer in an amount of 30-40 wt % with respect to the gelatin, and drying the shell until the water content becomes 5-10% by weight.

One manufacturing process used to make soft capsules uses a rotary die machine to encapsulate a fill material between two films. The rotary die method is more commonly referred to as the Scherer process. In this process, for example, two separate, continuous bands or sheets of gelatin are feed into the rotary die machine. The fill material or ingredients are simultaneously injected by an injector wedge between the two gelatin bands as the bands are drawn between two opposing, rotating dies or rollers. The rotating dies each have a plurality of cavities which align on opposing sides of the gelatin bands. The bands are pinched between the dies with each die cavity essentially forming one-half of a capsule. Thus, the gelatin bands and the fill material are introduced between the rotating dies where the fill material is sealed within the two halves of gelatin. Once formed, the gelatin capsule is ejected from the rotating die machine. Subsequent processes are used to prepare the gelatin capsule for packaging and shipment.

As used in this specification and in the claims, the term gelatin is meant to include not only the mammalian gelatin such as bovine and porcine, but also fish gelatins and other non-gelatin materials that are useful in soft capsule preparation. Those skilled in the art readily appreciate that there are a number of non-gelatin materials that can be used for soft capsule preparation such as modified starches and carrageenans, modified starches alone, and other compositions that are well known to those skilled in the art.

Gelatin is a substantially pure protein food ingredient, obtained by the thermal denaturation of collagen, which is the most common structural material and most common protein in animals. Gelatin forms thermally reversible gels with water, which gives gelatin products unique properties, such as reversible sol-gel transition states at near physiologic temperatures. Therefore, gelatin encapsulation of a fill material having an elevated temperature is problematic.

The temperature influence on the gelatin's physical properties imposes significant process challenges for encapsulating fill materials that are heated prior to the encapsulation process. This is particularly true when the fill material approaches, or exceeds, a gelatin sealing temperature. Capsules having hot fill materials readily deform when they make contact with external surfaces. The deformation is due to the elevated temperature of the fill material which maintains the gelatin at a temperature where the gelatin is very soft and pliable. While deformation, by itself, does not generally result in any deleterious problems with how the capsule functions, permanent deformation is unacceptable from a product aesthetics perspective. That is, consumers respond negatively to poor shape uniformity, finding faceted or flattened capsules unacceptable. Therefore, capsules that are deformed or that lack of shape uniformity are not merchantable.

The soft capsule manufacturing industry has long sought a softgel manufacturing processes that can encapsulate hot fill materials within gelatin. The numerous advantages of the gelatin capsule may be expanded by enlarging the variety of fill materials that may be encapsulated. In addition, there is a need for a manufacturing process that is capable of encapsulating hot fill materials at a high rate, yet can provide aesthetically pleasing, uniformly formed capsules which do not permanently deform during subsequent handling or packaging. Finally, there is a need for a softgel manufacturing process that is environmentally friendly, consumer safe, and cost effective. The present invention provides these aforementioned qualities by contacting the capsule with a chilled liquid immediately subsequent to capsule formation.

### SUMMARY OF THE INVENTION

In its most general configuration, the present invention advances the state of the art with a variety of new capabilities and overcomes many of the shortcomings of prior devices in new and novel ways. In its most general sense, the present invention overcomes the shortcomings and limitations of the prior art in any of a number of generally effective configurations. The instant invention demonstrates such capabilities and overcomes many of the shortcomings of prior methods in new and novel ways.

A primary mixing system may be used to mix, homogenize, and heat one or more fill materials. The fill material may be pumped to a secondary mixing system which heats the fill material to a fill material temperature prior to being fed to an encapsulation pump head assembly. The encapsulation pump head assembly may receive the fill material from the secondary mixing system. A pair of rotating dies presses the fill material between the first and second gelatin bands at the gelatin bands sealing temperature, thus forming a capsule. In one embodiment of the instant invention, the fill material temperature is higher than the sealing temperature.

Following formation, the capsule is brought into contact with a chilled liquid. The chilled liquid may be at a chilled liquid temperature that is less than the fill material temperature and the sealing temperature. In one embodiment of the instant invention, the gelatin is cooled to a handling temperature so that it is sufficiently durable preventing discernible faceting or flattening of the capsule during further processing.

In another embodiment of the instant invention, the chilled liquid may be a liquid deemed safe with respect to product contact by the Food and Drug Administration. In one particular embodiment, the chilled liquid is fractionated coconut oil. Once the capsule is substantially at the handling temperature, the chilled liquid is separated from the capsule. Following separation of the chilled liquid from the capsule, the capsule is transferred into a dryer basket. The dryer basket reduces the water content of the capsule so that the gelatin sheath is not substantially sticky.

In another embodiment of the instant invention, the capsule may contact a flowing chilled liquid layer. In yet another embodiment of the instant invention, the flowing chilled liquid layer discharges the capsule into a chilled liquid bath.

The system for cooling a hot-filled softgel capsule is designed to cool the capsule formed by the rotary die machine. As previously mentioned, the rotary die machine encases the fill material between two gelatin bands by sealing the gelatin bands together at the sealing temperature.

In one embodiment of the instant invention, a chilled liquid conveyor tray is filled with the chilled liquid. The chilled liquid conveyor tray is formed with a base, at least one sidewall, a chilled liquid influent port, and a discharge edge. The sidewall is connected to and surrounds a portion of the base. Thus, an interior surface and an exterior surface are formed. The chilled liquid influent port extends from the exterior surface to the interior surface to permit the chilled liquid to flow into the chilled liquid conveyor tray. The discharge edge connects the interior surface to the exterior surface so that the chilled liquid, carrying the capsule, may flow out of the chilled liquid conveyor tray.

The chilled liquid enters the chilled liquid conveyor tray through the chilled liquid influent port. The chilled liquid forms a flowing chilled liquid layer having a flowing chilled liquid layer depth and a liquid layer flow rate inside the chilled liquid conveyor tray. The capsule drops into contact with the flowing chilled liquid layer and heat flows from the capsule to the chilled liquid. The chilled liquid and the capsule flow across the discharge edge and out of the chilled liquid conveyor tray.

In another embodiment of the instant invention, the chilled liquid conveyor tray may include a chilled liquid layer forming base and the sidewall has a proximal side, a distal side, and a back side. A chilled liquid passageway is formed between the chilled liquid layer forming base and the base. The chilled liquid flows through a chilled liquid influent port into the chilled liquid passageway, through a chilled liquid layer forming passageway and onto a chilled liquid layer forming surface.

In another embodiment, the system further includes a chilled liquid tank filled with the chilled liquid. The chilled liquid tank holds a chilled liquid bath with flow of the chilled liquid supplied from the chilled liquid conveyor tray. In another embodiment of the instant invention, the system for cooling a hot-filled softgel capsule may include discharging the capsules directly into the chilled liquid tank filled with the chilled liquid.

Thus, there is disclosed a method of producing a hot-filled softgel capsule comprising the steps: encapsulating a fill material at a fill material temperature by injecting the fill material between a first gelatin band and a second gelatin band wherein the first gelatin band and the second gelatin band are sealed at a sealing temperature such that a capsule is formed; bringing the capsule into contact with a chilled liquid wherein the liquid is at a temperature less than the fill material temperature, and wherein said chilled liquid is a Food and Drug Administration approved liquid; cooling the capsule with the chilled liquid to a handling temperature such that the capsule does not substantially deform, wherein the handling temperature is less than the fill material temperature; and separating the capsule from the chilled liquid, which comprises blowing a pressurized gas onto the capsule.

There is further disclosed a system for cooling a hot-filled softgel capsule where a capsule is formed by encasing a fill material held at a fill material temperature between two gelatin bands sealed together at a sealing temperature, comprising: a chilled liquid conveyor tray formed with a base, at least one sidewall, a chilled liquid influent port, and a discharge edge, wherein the sidewall is connected to and surrounds a portion of the base thereby forming an interior surface and an exterior surface, the chilled liquid influent port extends from the exterior surface to the interior surface, and the discharge edge connects the interior surface to the exterior surface, wherein a chilled liquid enters the chilled liquid conveyor tray at a chilled liquid temperature through the chilled liquid influent port and forms a flowing chilled liquid layer having a flowing chilled liquid layer depth and a liquid layer flow rate, whereby the capsule contacts the flowing chilled liquid layer, heat flows from the capsule to the chilled liquid, and the discharge edge discharges the capsule and the chilled liquid out of the chilled liquid conveyor tray.

Various objects and advantages of the present invention will become apparent from the following detailed description when viewed in conjunction with the accompanying drawings, which set forth certain embodiments of the invention.

### BRIEF DESCRIPTION OF THE DRAWINGS

Without limiting the scope of the present invention as claimed below and referring now to the drawings and figures:

FIG. 1 is a schematic of an embodiment of the instant invention, not to scale;

FIG. 2 is an embodiment of the encapsulation assembly of the instant invention, not to scale;

FIG. 3 is a schematic of an embodiment of the flowing chilled liquid layer and an embodiment of the chilled liquid bath showing capsules being transported with the flowing chilled liquid layer to the chilled liquid bath, not to scale;

FIG. 4 is a perspective view of an embodiment of the chilled liquid conveyor tray, not to scale; and

FIG. 5 is a cross-sectional view taken along section line 5-5 in FIG. 4 of an embodiment of the chilled liquid conveyor tray.

### DETAILED DESCRIPTION OF THE INVENTION

The method for producing and the system for cooling a hot-filled softgel capsule of the instant invention enables a significant advance in the state of the art. The preferred embodiments of the apparatus accomplish this by new and novel arrangements of elements that are configured in unique and novel ways and which demonstrate previously unavailable but preferred and desirable capabilities. The detailed description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

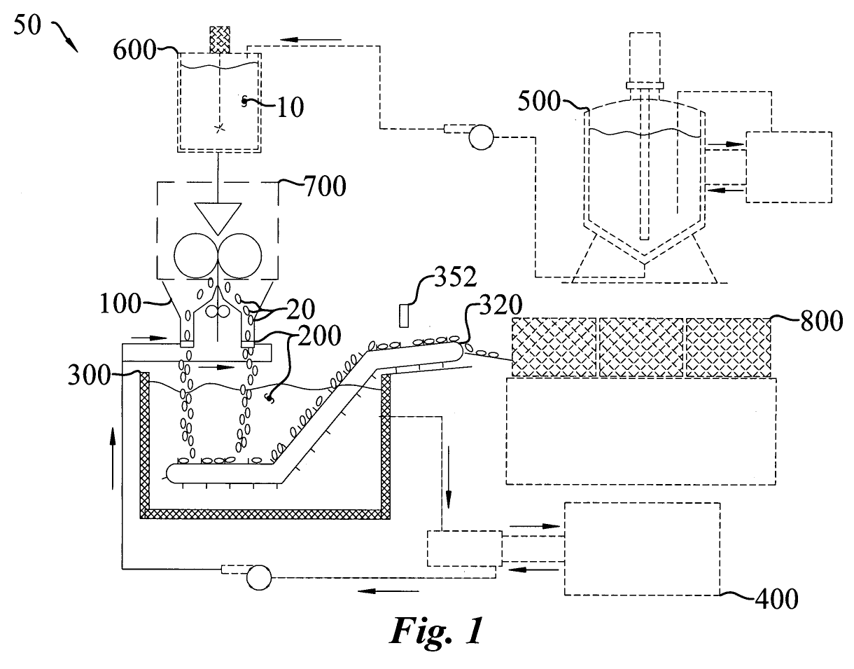

As seen in FIG. 1, the method for producing a hot-filled capsule may include a primary mixing system (500) used to mix and homogenize one or more fill materials (10). During the mixing and homogenization, the primary mixing system (500) heats the fill material (10) to an elevated temperature. For example, a heating bath may be coupled to a jacketed tank. A heated fluid is circulated from the heating bath to the tank to heat the fill material (10). As one skilled in the art will appreciate, the temperature may be controlled with a temperature sensing device coupled to a temperature controller which energizes a heat source.

With continued reference to FIG. 1, the fill material (10) is pumped to a secondary mixing system (600) which may, for example, be a transfer receiver. The secondary mixing system (600) may continue to perturb and heat the fill material (10) to a fill material temperature prior to being fed to an encapsulation pump head assembly (700). As one skilled in the art will appreciate, other means may be used to heat the fill material (10). Additionally, mixing the fill material (10) while heating may not be necessary. For example, the fill material (10) may be locally heated, but not mixed, immediately prior to entering the encapsulation pump head assembly (700).

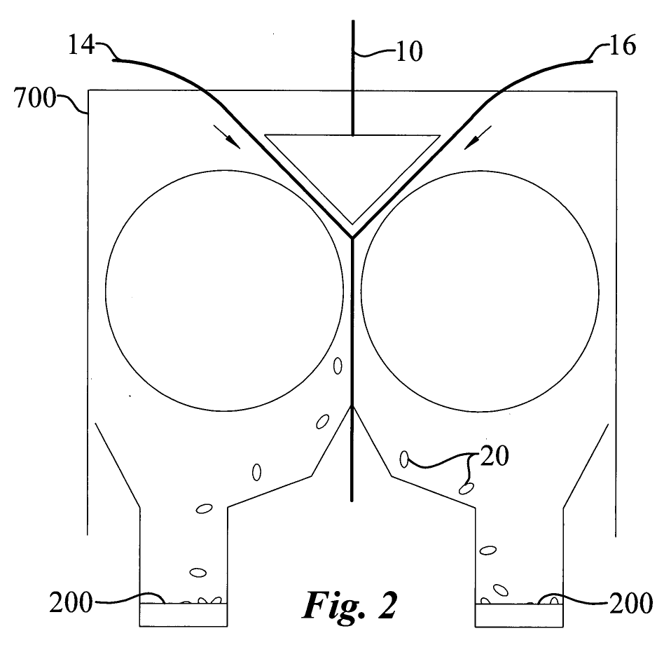

The encapsulation pump head assembly (700) is best seen in FIG. 2. In this embodiment, the encapsulation pump head assembly (700) may receive the fill material (10) from the secondary mixing system (600) together with a first gelatin band (14) and a second gelatin band (16). A pair of rotating dies encapsulates the fill material (10) between the first and second gelatin bands (14, 16) forming a capsule (20) where the fill material (10) is surrounded by gelatin. As one skilled in the art will observe and appreciate, encapsulating the fill material (10) between the first and second gelatin bands (14, 16) may require the gelatin to be held at a sealing temperature to seal each half capsule to the other in order to form the capsule (20). In one embodiment of the instant invention, the fill material temperature is approximately the same as the sealing temperature. In one particular embodiment, the fill material temperature is between approximately 38 degrees Celsius and approximately 45 degrees Celsius. As the fill material temperature surpasses the sealing temperature, the gelatin becomes progressively softer, that is, the gelatin viscosity decreases, thus making uniform, aesthetic capsule formation more difficult. As one skilled in the art will observe and appreciate, gelatin viscosity may be a function of a number of factors, including the type of gelatin and the temperature. For example, pork, bovine, and fish gelatins do not exhibit the same viscosity relationship with temperature.

With reference, once again to FIG. 1, in this embodiment of the instant invention, once formed, the capsule (20) is brought into contact with a chilled liquid (200). The chilled liquid (200) is at a chilled liquid temperature. As one skilled in the art will observe and appreciate, when the chilled liquid temperature is less than the sealing temperature and the fill material temperature, heat is transferred from the capsule (20) to the chilled liquid (200) causing the temperature of the capsule (20) to decrease and the chilled liquid temperature to increase. In one embodiment of the instant invention, the chilled liquid temperature is between approximately minus 10 degrees Celsius and approximately 10 degrees Celsius. However, the chilled liquid temperature may be only slightly less than the sealing temperature or the chilled liquid temperature may be colder than minus 10 degrees Celsius. In either case, any temperature difference between the chilled liquid (200) and the capsule (20) that cools the capsule (20) may be sufficient to prevent permanent deformation. For example, as the temperature difference between the fill material (10) and the chill liquid (200) increases, the cooling rate of the capsule (20) increases. Large capsules may require higher cooling rates to bring them from the fill material temperature to a handling temperature within a sufficient time period to make their manufacture cost effective. The chilled liquid temperature may be adjusted by setting a target temperature on a chilled liquid cooling system (400), best seen in FIG. 1. Furthermore, by maintaining the gelatin sheath at the handling temperature, the capsule (20) may resist external pressures exerted on the capsule (20). Thus, the capsule (20) is less likely to form facets or flat spots as a result of contact with external objects.

In one embodiment of the instant invention, the chilled liquid (200) is a Food and Drug Administration approved non-aqueous liquid deemed safe for human consumption. In one particular embodiment, the chilled liquid (200) is fractionated coconut oil. Other representative non-aqueous edible liquids suitable for chilling in the present invention include oils such as linseed oil, sesame oil, mustard oil, castor oil, clove oil, and vegetable and marine oils. In general, any material that does not degrade or dissolve the soft capsule, is relatively inexpensive, non-toxic, and easily removed from the soft capsule is suitable for use in the present invention.

Once the capsule (20) is substantially at the handling temperature, the chilled liquid (200) is separated from the capsule (20). In one embodiment of the instant invention, a large percentage of the chilled liquid (200) is removed from the capsule (20) with an air knife (352). The air knife (352) forms a high pressure gas stream and directs the gas stream onto the capsule (20). In one particular embodiment, the gas stream is between approximately 10 pounds per square inch (psi) and approximately 60 psi. As seen in FIG. 1, in another embodiment of the instant invention, following separation of the chilled liquid (200) from the capsule (20), the capsule (20) is transferred into a dryer basket (800). The dryer basket (800) reduces the water content of the capsule (20). As one skilled in the art will observe and appreciate, numerous drying baskets may be implemented, depending on the water volume desired, the production rate, and the capsule size, to name only a few factors. In one embodiment of the invention, for example the embodiment seen in FIG. 1, successful production of capsules of the size range #4 to #40 with any one or more of the common shapes, such as round, oval, or oblong with heated fill materials, is possible.

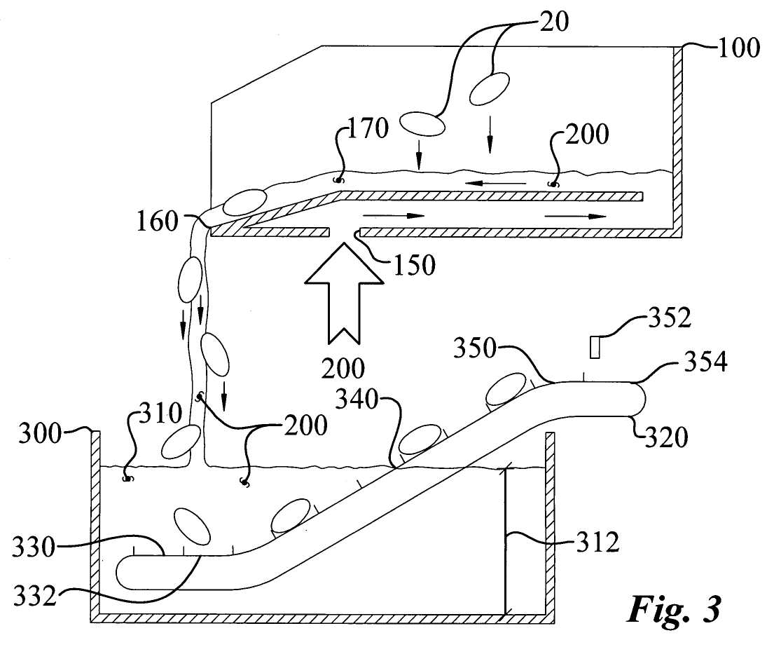

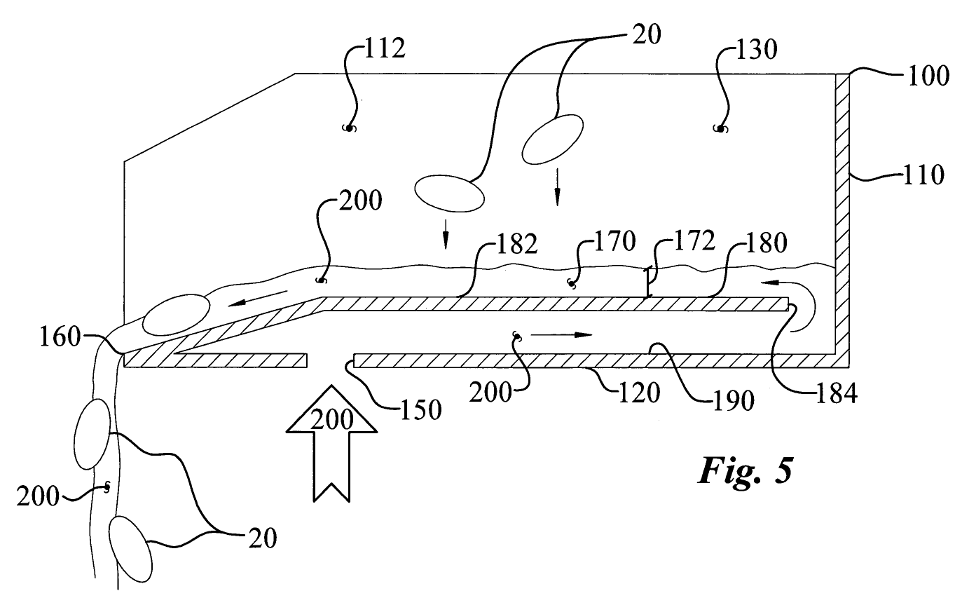

In another embodiment, as seen in FIGS. 3 and 5, the chilled liquid (200) may take the form of a flowing chilled liquid layer (170). The flowing chilled liquid layer (170) is the chilled liquid (200) formed into a flowing layer having a flowing liquid layer depth (172) and a flowing liquid layer flow rate. As one skilled in the art will observe, when the capsule (20) contacts the flowing chilled liquid layer (170) heat is transferred from the capsule (20) to the chilled liquid (200). In addition, while cooling the capsule (20), the flowing chilled liquid layer (170) transports the capsule (20). In one particular embodiment of the instant invention, the flowing liquid layer depth is between approximately 0.5 inches and approximately 2 inches. As the capsule size increases the flowing liquid layer depth (172) may also increase to help cushion the capsule (20) as is falls from the encapsulation pump head assembly (700) following formation. In another embodiment of the instant invention, the flowing liquid layer flow rate is between approximately 1 gallon per minute and approximately 30 gallons per minute depending on the flowing liquid layer depth (172) desired. Again, the capsule size may determine the liquid layer flow rate. As with the flowing liquid layer depth (172), one skilled in the art will appreciate that having a higher flowing, liquid layer flow rate will generally provide a deeper flowing liquid layer depth (172).

With reference to FIG. 3, in another embodiment of the instant invention, the flowing chilled liquid layer (170) discharges the capsule (20) into a chilled liquid bath (310) having a chilled liquid bath depth (312). Once the capsule (20) departs the flowing chilled liquid layer (170), the capsule (20) may be submerged in the chilled liquid bath (310) where heat is transferred from the capsule (20) to the chilled liquid bath (310). Similar to the flowing liquid layer depth (172), the chilled liquid bath depth (312) may increase, as the capsule size increases and as the fill material temperature increases, in order to provide sufficient cooling to the capsule (20) and to prevent the capsule (20) from deforming due to contact between the capsule (20) and another capsule or rigid surface.

In another embodiment, immediately after the capsule (20) is formed by the encapsulation pump head assembly (700), the capsule (20) is brought into contact with the chilled liquid bath (310), as seen in FIGS. 1 and 3, held at a chilled liquid bath temperature. The chilled liquid bath temperature is less than the fill material temperature so that when the capsule (20) contacts the chilled liquid bath (310) heat is transferred from the capsule (20) to the chilled liquid bath (310).

In one embodiment of the instant invention, a temperature drop from the fill material temperature to the handling temperature may be as little as 8 degrees Celsius for small capsules to bring them to the handling temperature. In another embodiment, the capsule (20) may require a temperature drop of at least 34 degrees Celsius. The capsule size also influences the cooling period required. Therefore, in one embodiment of the instant invention, the cooling period may be between approximately 30 seconds and approximately 120 seconds, depending on the capsule size, fill material temperature, capsule production rate, and the chilled liquid temperature. As one skilled in the art will appreciate, as the capsule size increases, the thermal mass of the fill material (10) increases relative to the mass of the gelatin. In turn, as the fill material thermal mass increases, the cooling period may increase in order to remove additional thermal energy to bring the capsule (20) to the handling temperature.

The system for cooling a hot-filled softgel capsule (50) may be designed to cool the capsule (20) formed by the rotary die machine. As previously mentioned and as seen in FIG. 2, the rotary die machine encases the fill material (10) between two gelatin bands by sealing the gelatin bands together at the sealing temperature.

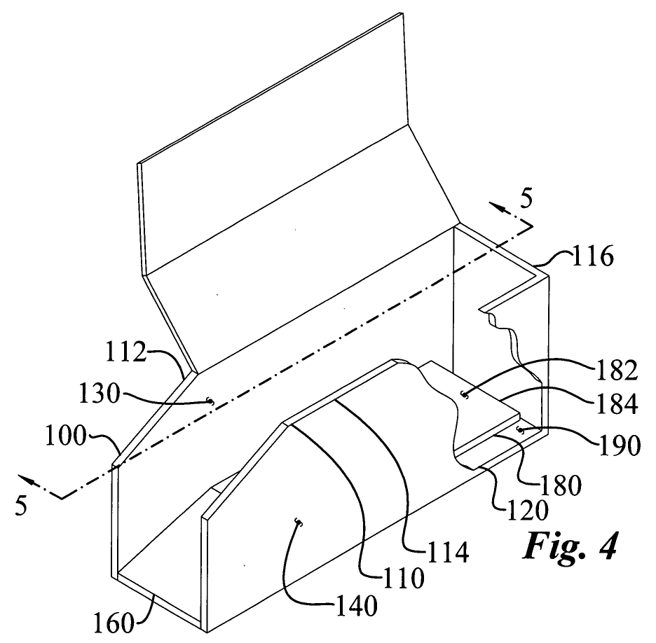

As seen in FIGS. 4 and 5, in one embodiment of the instant invention, a chilled liquid conveyor tray (100) is filled with the chilled liquid (200). The chilled liquid conveyor tray (100) is formed with a base (120), at least one sidewall (110), a chilled liquid influent port (150), and a discharge edge (160). The sidewall (110) is connected to and surrounds a portion of the base (120). Thus, an interior surface (130) and an exterior surface (140) are formed. The chilled liquid influent port (150) extends from the exterior surface (140) to the interior surface (130) to permit the chilled liquid (200) to flow into the chilled liquid conveyor tray (100). The discharge edge (160) connects the interior surface (130) to the exterior surface (140) so that the chilled liquid (200) may flow out of the chilled liquid conveyor tray (100). As one skilled in the art will observe and appreciate, the chilled liquid conveyor tray (100) may be designed to allow the chilled liquid (200) flow in a laminar or turbulent fashion. For example, various devices or structure may be added to the chilled liquid conveyor tray (100) to agitate the chilled liquid (200) thus creating a turbulent flow pattern within the chilled liquid conveyor tray (100). On the other hand, the dimensions of the chilled liquid conveyor tray (100) and the chilled liquid flow may be adjusted to provide laminar flow of the chilled liquid (200) within the chilled liquid conveyor tray (100). One skilled in the art will also observe that the length of the chilled liquid conveyor tray (100) may be designed to target a length of time the capsule (20) resides in the chilled liquid conveyor tray (100). Besides the length, the declination of the chilled liquid conveyor tray (100) may provide another means to control the length of time the capsule (20) spends in the chilled liquid conveyor tray (100).

During operation, as best seen in FIG. 5, the chilled liquid (200) enters the chilled liquid conveyor tray (100) through the chilled liquid influent port (150). The chilled liquid (200) forms the flowing chilled liquid layer (170) having the flowing chilled liquid layer depth (172) and the liquid layer flow rate inside the chilled liquid conveyor tray (100). Once formed, the capsule (20) drops into contact with the flowing chilled liquid layer (170). Heat flows from the capsule (20) to the chilled liquid (200) while the capsule (20) is transported to the discharge edge (160). The chilled liquid (200) and the capsule (20) flow across the discharge edge (160) and out of the chilled liquid conveyor tray (100).

As one skilled in the art will observe and appreciate, the chilled liquid conveyor tray (100) may have many configurations and accomplish cooling of the capsule (20) subsequent to its formation. For example, the chilled liquid influent port (150) may be located in the sidewall (110) rather than in the base (120). In another example, the discharge edge (160) may be elevated from the base (120) forming a shallow weir to aide in the formation of the flowing chilled liquid layer (170). In addition, the chilled liquid conveyor tray (100) may be formed from a variety of materials. By way of example and not limitation, the chilled liquid conveyor tray (100) may be made of stainless sheet metal or plastic.

In another embodiment of the instant invention, the chilled liquid conveyor tray (100) may be designed to fit to an existing rotary die machine. As seen in FIGS. 4 and 5, the chilled liquid conveyor tray (100) may include a chilled liquid layer forming base (180) and the sidewall (110) has a proximal side (112), a distal side (114), and a back side (116). The chilled liquid layer forming base (180) extends from the proximal side (112) to the distal side (114) of the sidewall (110). A chilled liquid passageway (190) is formed between the chilled liquid layer forming base (180) and the base (120). The chilled liquid layer forming base (180) has a chilled liquid layer forming surface (182) and a chilled liquid layer forming passageway (184). The chilled liquid passageway (190) provides fluid communication between the chilled liquid influent port (150) and the chilled liquid layer forming passageway (184), as best seen in FIG. 5. Thus, the chilled liquid (200) flows through the chilled liquid influent port (150) into the chilled liquid passageway (190). The chilled liquid (200) then flows through the chilled liquid layer forming passageway (184) and onto the chilled liquid layer forming surface (182) where the flowing chilled liquid layer (170) is formed.

In another embodiment, the system (50) further includes a chilled liquid tank (300) filled with the chilled liquid (200), as seen in FIG. 3. The chilled liquid tank (300) holds a chilled liquid bath (310) that is in fluid communication with the chilled liquid conveyor tray (100) via the discharge edge (160). During operation, the chilled fluid (200) and the capsule (20) flow from the chilled liquid conveyor tray (100) to the chilled liquid tank (300). The chilled liquid tank (300) has a capsule transfer conveyor (320) having a transfer conveyor submerged portion (330), a transfer conveyor inclined portion (340), and a transfer conveyor chilled liquid removal portion (350).

The transfer conveyor submerged portion (330) captures the capsule (20) on a capsule capturing portion (332) as the capsule (20) falls through the chilled liquid (200). The transfer conveyor inclined portion (340) transports the capsule (20) out of the chilled liquid bath (310) to the transfer conveyor chilled liquid removal portion (350) where a portion of the chilled liquid (200) is removed. The transfer conveyor chilled liquid removal portion (350) may have the air knife (352) positioned to direct pressurized gas onto the capsules (20). The air knife (352) cleans a portion of the chilled liquid (200) from the capsule (20). The transfer conveyor chilled liquid removal portion (350) may have a discharge end (354). The capsule (20) is transported off the capsule transfer conveyor (320) at a capsule discharge end (354). As one skilled in the art will observe and appreciate, the transfer conveyor inclined portion (340) may be designed to transport the capsules (20) vertically out of the chilled liquid bath (310) rather than at along an inclination, as seen in FIGS. 1 and 3.

As one skilled in the art will observe and appreciate, the cooling period may be adjusted by altering the depth of the chilled liquid bath (310) and the velocity of the capsule transfer conveyor (320). By increasing the depth of the chilled liquid bath (310) or by decreasing the velocity of the capsule transfer conveyor (320), the cooling period may be increased. As one skilled in the art will observe, even while the capsule (20) is in contact with the capsule transfer conveyor (320), the capsule (20) may not deform even though the fill material (10) may still be hot. In addition to providing a means for rapidly transferring heat from the capsule (20), when the capsule (20) is submerged in the chilled liquid (200), the chilled liquid (200) provides buoyancy to the capsule (20). Thus, the weight of the capsule (20) does not rest entirely on the capsule contact area with transfer conveyor (320) until the capsule (20) is removed from the chilled liquid (200) at which point it has been cooled to the handling temperature. The cooling period may require adjustment depending upon the capsule size, the fill material temperature, and the production rate.

In another embodiment of the instant invention, by redesigning the encapsulation pump head assembly (700), the system for cooling a hot-filled softgel capsule (50) may include discharging the capsules (20) directly into the chilled liquid tank (300) filled with the chilled liquid (200). Similar to an embodiment of the instant invention having both the chilled liquid conveyor tray (100) and the chilled liquid tank (300), the chilled liquid tank (300) may have the capsule transfer conveyor (320) having the transfer conveyor submerged portion (330), the transfer conveyor inclined portion (340), and the transfer conveyor chilled liquid removal portion (350).

In one embodiment of the instant invention, the liquid layer flow rate is between approximately 1 gallon per minute and 30 gallons per minute. The liquid layer flow rate may be adjusted to account for the productivity of the encapsulation machine, the capsule size, the temperature of the fill material, the dimensions of the chilled liquid conveyor tray (100), and the chilled liquid layer depth (172).

By way of example and not limitation, in one embodiment of the instant invention, a #40 capsule is produced with the fill material temperature of at least 38 degrees Celsius. After leaving the encapsulation pump head assembly (700), the capsule (20) drops into the liquid conveyor tray (100). The chilled liquid (200) is fractionated coconut oil held at a temperature of approximately 0 degrees Celsius. The capsule (20) is cooled as the capsule (20) is transported across the discharge edge (160) out of the chilled liquid conveyor tray (100) and into the chilled liquid bath (310). The capsule (20) sinks and gently contacts the capsule transfer conveyor (320). The capsule transfer conveyor (320) transports the capsule (20) out of the chilled liquid (200) to the air knife (352) where the majority of the chilled liquid (200) is removed. The cooling period from the capsule (20) first contact with the chilled liquid (200) to exiting the chilled liquid bath (310) is approximately 60 seconds. Moreover, no permanent deformation is apparent in the #40 capsule.

In another example, the fill material temperature is greater than approximately 35 degrees Celsius. Following encapsulation where the gelatin is sealed around the fill material (10), the capsule (20) is dropped into the chilled liquid conveyor tray (100). The chilled liquid temperature is less than approximately 10 degrees Celsius. The capsule (20) is transported into the chilled liquid bath (310) and emerges between approximately 30 seconds and 60 seconds later. In another example, the fill material temperature is at least approximately 38 degrees Celsius and the chilled liquid temperature is less than approximately 0 degrees Celsius. Generally, as the fill material temperature increases, the chilled liquid temperature decreases.

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and/or additional or alternative materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims.

### INDUSTRIAL APPLICABILITY

The system for producing a hot-filled softgel capsule answers a long felt need for a system and method that is capable of encapsulating hot fill material in gelatin. The system is used to produce small or large softgel capsules of various shapes by injecting the heated fill material between two bands of gelatin introduced between two rotating dies. The present invention discloses a system and method that implements a chilled liquid subsequent to encapsulation. The softgel capsules produced by the rotating dies contact the chilled liquid thus transferring heat from the capsule to the chilled liquid. The system and method thereby avoids some of the aesthetic problems associated with encapsulating hot fill materials with gelatin. The system of the present invention produces softgel capsules that are safe for consumers, and the system is environmentally friendly and cost effective.

We claim:

1. A method of producing a hot-filled softgel capsule comprising the steps of:
encapsulating a fill material (10) at a fill material temperature by injecting the fill material between a first gelatin band (14) and a second gelatin band (16) wherein the first gelatin band (14) and the second gelatin band (16) are sealed at a sealing temperature such that a capsule (20) is formed;
bringing the capsule (20) into contact with a chilled liquid (200) wherein the liquid (200) is at a temperature less than the fill material temperature, and wherein said chilled liquid (200) is a Food and Drug Administration approved liquid;
cooling the capsule (20) with the chilled liquid (200) to a handling temperature such that the capsule (20) does not substantially deform, wherein the handling temperature is less than the fill material temperature; and
separating the capsule (20) from the chilled liquid (200), which comprises blowing a pressurized gas onto the capsule (20).

2. The method of producing a hot-filled softgel capsule of claim 1, wherein the chilled liquid (200) is fractionated coconut oil.

3. The method of producing a hot-filled softgel capsule of claim 1, wherein the fill material temperature is greater than approximately 35 degrees Celsius and the chilled liquid temperature is less than approximately 10 degrees Celsius.

4. The method of producing a hot-filled softgel capsule of claim 1, wherein the fill material temperature is at least approximately 38 degrees Celsius and the chilled liquid temperature is less than approximately 0 degrees Celsius.

5. The method of producing a hot-filled softgel capsule of claim 1, wherein the chilled liquid temperature is between approximately minus 10 degrees Celsius and approximately 10 degrees Celsius.

6. The method of producing a hot-filled softgel capsule of claim 1, wherein the step of bringing the capsule (20) into contact with the chilled liquid (200) further includes bringing the capsule (20) into contact with a flowing chilled liquid layer (170), wherein the capsule (20) contacts the flowing chilled liquid layer (170) and heat is transferred from the capsule (20) to the chilled liquid (200) while the flowing chilled liquid layer (170) transports the capsule (20).

7. The method of producing a hot-filled softgel capsule of claim 6, wherein the flowing chilled liquid layer (170) flows into a chilled liquid bath (310) thereby totally immersing the capsule (20) in the chilled liquid bath (310) and permitting the transfer of heat from the capsule (20) to the chilled liquid bath (310).

8. The method of producing a hot-filled softgel capsule of claim 1, wherein the step of bringing the capsule (20) into contact with the chilled liquid (200) further includes bringing the capsule (20) into contact with a chilled liquid bath (310) thereby totally immersing the capsule (20) in the chilled liquid bath (310) and permitting the transfer of heat from the capsule (20) to the chilled liquid bath (310).

9. The method of producing a hot-filled softgel capsule of claim 1, wherein the temperature drop from the fill material temperature to the handling temperature is at least 34 degrees Celsius and occurs over a cooling period of between approximately 30 seconds and approximately 120 seconds.

10. A system for cooling a hot-filled softgel capsule (50) where a capsule (20) is formed by encasing a fill material (10) held at a fill material temperature between two gelatin bands sealed together at a sealing temperature, comprising:
a chilled liquid conveyor tray (100) formed with a base (120), at least one sidewall (110), a chilled liquid influent port (150), and a discharge edge (160), wherein the sidewall (110) is connected to and surrounds a portion of the base (120) thereby forming an interior surface (130) and an exterior surface (140), the chilled liquid influent port (150) extends from the exterior surface (140) to the interior surface (130), and the discharge edge (160) connects the interior surface (130) to the exterior surface (140), wherein a chilled liquid (200) enters the chilled liquid conveyor tray (100) at a chilled liquid temperature through the chilled liquid influent port (150) and forms a flowing chilled liquid layer (170) having a flowing chilled liquid layer depth (172) and a liquid layer flow rate, whereby the capsule (20) contacts the flowing chilled liquid layer (170), heat flows from the capsule (20) to the chilled liquid (200), and the discharge edge (160) discharges the capsule (20) and the chilled liquid (200) out of the chilled liquid conveyor tray (100).

11. The system for cooling a hot-filled softgel capsule (50) of claim 10, wherein the chilled liquid conveyor tray (100) further includes a chilled liquid layer forming base (180) and the sidewall (110) has a proximal side (112), a distal side (114), and a back side (116), wherein,
(A) the chilled liquid layer forming base (180) extends from the proximal side (112) to the distal side (114) of the sidewall (110) thereby forming a chilled liquid passageway (190) between the chilled liquid layer forming base (180) and the base (120), and
(B) the chilled liquid layer forming base (180) has a chilled liquid layer forming surface (182) and a chilled liquid layer forming passageway (184), wherein,
(i) the chilled liquid passageway (190) provides fluid communication between the chilled liquid influent port (150) and the chilled liquid layer forming passageway (184), whereby the chilled liquid (200) flows through the chilled liquid influent port (150) into the chilled liquid passageway (190), and
(ii) the chilled liquid layer forming passageway (184) places the chilled liquid passageway (190) in fluid communication with the chilled liquid layer forming surface (182), whereby the flowing chilled liquid layer (170) is formed on the chilled liquid layer forming surface (182) by flowing through the chilled liquid layer forming passageway (184).

12. The system for cooling a hot-filled softgel capsule (50) of claim 10, further including a chilled liquid tank (300) containing the chilled liquid (200) thereby creating a chilled liquid bath (310), wherein
(A) the discharge edge (160) is positioned relative to the chilled liquid bath (310) so that the chilled fluid (200) and the capsule (20) flow from the chilled liquid conveyor tray (100) to the chilled liquid tank (300); and
(B) the chilled liquid tank (300) has a capsule transfer conveyor (320) having a transfer conveyor submerged portion (330), a transfer conveyor inclined portion (340), and a transfer conveyor chilled liquid removal portion (350); wherein
(i) the transfer conveyor submerged portion (330) captures the capsule (20) as the capsule (20) falls through the chilled liquid (200),
(ii) the transfer conveyor inclined portion (340) transports the capsule (20) out of the chilled liquid bath (310), and
(iii) the transfer conveyor chilled liquid removal portion (350) has a chilled liquid removal device (352) and a discharge end (354), wherein the chilled liquid removal device (352) cleans a portion of the chilled liquid (200) from the capsule (20) and the capsule (20) is transported off the capsule transfer conveyor (320) at the capsule discharge end (354).

13. The system for cooling a hot-filled softgel capsule (50) of claim 11, further including a chilled liquid tank (300) containing the chilled liquid (200) thereby creating a chilled liquid bath (310), wherein
(A) the discharge edge (160) is positioned relative to the chilled liquid bath (310) so that the chilled fluid (200) and the capsule (20) flow from the chilled liquid conveyor tray (100) to the chilled liquid tank (300); and
(B) the chilled liquid tank (300) has a capsule transfer conveyor (320) having a transfer conveyor submerged portion (330), a transfer conveyor inclined portion (340), and a transfer conveyor chilled liquid removal portion (350), wherein
(i) the transfer conveyor submerged portion (330) captures the capsule (20) as the capsule (20) falls through the chilled liquid (200),
(ii) the transfer conveyor inclined portion (340) transports the capsule (20) out of the chilled liquid bath (310), and
(iii) the transfer conveyor chilled liquid removal portion (350) has a chilled liquid removal device (352) and a discharge end (354), wherein the chilled liquid removal device (352) cleans a portion of the chilled liquid (200) from the capsule (20) and the capsule (20) is transported off the capsule transfer conveyor (320) at the capsule discharge end (354).

14. The system for cooling a hot-filled softgel capsule (50) of claim 10, wherein the chilled liquid layer depth (172) is between approximately 0.5 inches and approximately 2 inches.

15. The system for cooling a hot-filled softgel capsule (50) of claim 10, wherein the liquid layer flow rate is between approximately 1 gallon per minute and approximately 30 gallons per minute.

16. The method of cooling a hot-filled softgel capsule of claim 10, wherein the chilled liquid removal device (352) is an air knife which blows pressurized gas onto the capsule (20) to substantially remove the chilled liquid (200).

17. A system for cooling a hot-filled softgel capsule (50) where a capsule (20) is formed by encasing a fill material (10) held at a fill material temperature between two gelatin bands sealed together at a sealing temperature, comprising:
a chilled liquid tank (300) filled with the chilled liquid (200) thereby creating a chilled liquid bath (310) at a chilled liquid bath temperature, wherein the capsule (20)
(i) drops into the chilled liquid bath (310),
(ii) sinks, and
(iii) transfers heat to the chilled liquid bath (310) because the chilled liquid bath temperature is less than the fill material temperature, and the chilled liquid tank (300) has a capsule transfer conveyor (320) for controlling the egress of the capsule (20) from the chilled liquid tank (300), wherein the capsule transfer conveyor (320) has a transfer conveyor submerged portion (330), a transfer conveyor inclined portion (340), and a transfer conveyor chilled liquid removal portion (350), and wherein,
(a) the transfer conveyor submerged portion (330) captures the capsule (20) as the capsule (20) falls through the chilled liquid (200),
(b) the transfer conveyor inclined portion (340) transports the capsule (20) out of the chilled liquid bath (310), and
(c) the transfer conveyor chilled liquid removal portion (350) has a chilled liquid removal device (352) and a discharge end (354), wherein the chilled liquid removal device (352) cleans a portion of the chilled liquid (200) from the capsule (20) and the capsule (20) is transported off the capsule transfer conveyor (320).

18. The method of cooling a hot-filled softgel capsule of claim 17, wherein the chilled liquid removal device (352) is an air knife which blows pressurized gas onto the capsule (20) to substantially remove the chilled liquid (200).
